(19) United States
(12) Patent Application Publication
(10) Pub. No.: US 2013/0045269 A1
(43) Pub. Date: Feb. 21, 2013

(54) FORMULATIONS AND METHODS FOR WOUND TREATMENT

(71) Applicant: Hossein DOVLATABADI, Birmingham, AL (US)

(72) Inventor: Hossein DOVLATABADI, Birmingham, AL (US)

(73) Assignee: Sorush LLC, Birmingham, AL (US)

(21) Appl. No.: 13/212,918

(22) Filed: Aug. 18, 2011

(51) Int. Cl. A61K 9/70; A61P 17/02; A61K 36/185

(52) U.S. Cl. 424/445; 424/769

(57) ABSTRACT

The present application relates to formulations and methods for treating wounds, including chronic wounds. Suitably, the formulations comprise antibiotics and lawsonia inermis extract in various amounts. In embodiments, the formulations are formulated as sprays or gels that can be applied directly to the wound or to a wound dressing that is then applied to the wound.

### BACKGROUND OF THE INVENTION

1. Field of the Invention

The present application relates to formulations and methods for treating wounds, including chronic wounds. Suitably, the formulations comprise antibiotics and lawsonia inermis extract in various amounts. In embodiments, the formulations are formulated as sprays or gels that can be applied directly to the wound or to a wound dressing that is then applied to the wound.

2. Background of the Invention

A skin wound is defined as a breach in the continuity of any body tissue caused by a direct injury to the skin. Approximately 5-7 million Americans are afflicted with chronic skin wounds that account for billions of dollars in medical expenses each year. (See Petrie et al., Surg Clin North Am 83:597-616 (2003); Frykberg et al., J. American college of Foot and Ankle Surgeons 39:S1-60 (2000), the disclosures of each of which are incorporated by reference herein in their entireties.) The incidence of chronic wounds is expected to increase dramatically due to an increased elderly population and incidence of diabetes, a disease that is accompanied by wound-healing deficiencies. (See Harrington et al., Diabetes Care 23:13334 (2000); Medina et al., J Burn Care Rehabil. 26: 306-19 (2005), the disclosures of each of which are incorporated by reference herein in their entireties.)

Commonly used approaches to treat chronic or acute wounds are typically based on simple wound care regimens involving debridement, cleaning, and application of moist dressings. (See Thomas S, Leigh 1 M. Wound dressings. In: Leaper D J, Harding K G, editors. Wounds: biology and management. New York, N.Y.: Oxford University Press, 1998: 166-82. (1998), the disclosure of each of which are incorporated by reference herein in their entireties.) Acceleration of wound closure not only results in decreased patient suffering and cost of wound treatment but may also minimize scarring and lead to formation of a more stable closed wound.

There exists therefore a need for a wound treatment formulation that is effective in generating rapid healing of wounds, including chronic wounds,

### SUMMARY OF PREFERRED EMBODIMENTS

The needs identified above are met by the present application providing methods and formulations for wound treatment.

In embodiments, topical formulations are provided. Exemplary topical formulations comprise about 0.0050% to about 0.050% polymyxin B, about 0.050% to about 0.50% clindamycin, about 0.050% to about 0.50% gentamicin, about 5% to about 15% lawsonia inermis extract and water.

In embodiments, the topical formulations are formulated as a spray, and in other embodiments the formulations can further comprise carboxymethylcellulose sodium salt, formulated as a gel.

Suitably, the topical formulations have a pH in the range of about pH 4.5 to about 5.5, suitably about pH 5.0.

In further embodiments, the topical formulations comprise about 0.0080% to about 0.0130% polymyxin B, about 0.080% to about 0.130% clindamycin, about 0.080% to about 0.130% gentamicin, about 8.0% to about 13.0% lawsonia inermis extract and water.

Also provided are topical spray formulations for treatment of a wound, Such formulations suitably comprise about 0.0080% to about 0.0130% polymyxin B, about 0.080% to about 0.130% clindamycin, about 0.080% to about 0.130% gentamicin, about 8.0% to about 13.0% lawsonia inermis extract and water. Exemplary topical gel formulations further comprise carboxymethylcellulose sodium salt.

Also provided are methods of treating a skin wound comprising applying to the wound a topical formulation comprising about 0.0050% to about 0.050% polymyxin B, about 0.050% to about 0.50% clindamycin, about 0.050% to about 0.50% gentamicin, about 5.0% to about 15.0% lawsonia inermis extract and water.

Suitably, the formulation is formulated as a spray or gel and applied directly to the wound. In other embodiments, the formulation is formulated as a spray or gel and applied to a wound dressing and then the dressing is applied to the wound. In embodiments, the formulation is applied to the wound at least once per day.

Suitably, the methods are useful for treating chronic wounds, including decubitus ulcers, diabetic ulcers or venous stasis ulcers. The formulations are also useful for treating post-surgical wounds.

Further embodiments, features, and advantages of the embodiments, as well as the structure and operation of the various embodiments, are described in detail below with reference to accompanying drawings.

### BRIEF DESCRIPTION OF THE FIGURES

FIGS. 1A-1C show the results of treatment of a foot amputation wound using an exemplary formulation described herein.

FIGS. 2A-2F show the results of treatment of a hysterectomy wound using an exemplary formulation described herein.

### DETAILED DESCRIPTION OF PREFERRED EMBODIMENTS

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. It should be understood that use of the term “about” also includes the specifically recited amount.

Technical and scientific terms used herein have the meaning commonly understood by one of skill in the art to which the present application pertains, unless otherwise defined. Reference is made herein to various methodologies and materials known to those of skill in the art.

In embodiments, the application provides topical formulations. The topical formulations suitably comprise the following active ingredients, polymyxin B, clindamycin, gentamicin and lawsonia inermis extract.

As used herein, a “topical formulation” refers to a composition that is administered topically to the surface of the skin. The topical formulations can be administered to any surface of the skin, including the face, arms, back, trunk, legs, chest, feet, hands, etc., as well as the inner surface of the mouth, nasal passages, tongue, vaginal walls, etc.

Polymyxin B is an antibiotic primarily used for treatment of resistant gram-negative infections. It is derived from the bacterium Bacillus polymyxa, and is a mixture of two closely related compounds, polymyxin B1 and polymyxin B2

Clindamycin is a lincosamide antibiotic, often used to treat infections with anaerobic bacteria.

Gentamicin is an aminoglycoside antibiotic, often use to treat Gram-negative organisms.

While the amounts of the various agents in the topical formulations can be varied by those of ordinary skill in the art to obtain various changes in the properties of the formulations, suitably the amounts of the agents are described below.

Unless otherwise indicated, all percentages of the agents described herein refer to weight percent (wt %) based upon the entire weight of the formulation,

In embodiments, suitably the topical formulations comprise about 0.0010% to about 0.10% polymyxin B. More suitably, the formulations comprise about 0.0050% to about 0.050% polymyxin B, or about 0.0080% to about 0.050% polymyxin B, about 0.0080% to about 0.040% polymyxin B, about 0.0080% to about 0.030% polymyxin B, about 0.0080% to about 0.020% polymyxin B, about 0.008% to about 0.013% polymyxin B, about 0.008% to about 0.012% polymyxin B or about 0.0080% polymyxin B, about 0.0085% polymyxin B, about 0.0090% polymyxin B, about 0.0095% polymyxin B, about 0.0096% polymyxin B, about 0.0097% polymyxin B, about 0.0098% polymyxin B, about 0.0099% polymyxin B, about 0.010% polymyxin B, about 0.011% polymyxin B, about 0.012% polymyxin B, about 0.013% polymyxin B, about 0.014% polymyxin B, about 0.015% polymyxin B, about 0.016% polymyxin B, about 0.017% polymyxin B, about 0.018% polymyxin B, about 0.019% polymyxin B, or about 0.020% polymyxin B, including any value or range between these values.

In embodiments, suitably the topical formulations comprise about 0.010% to about 1.0% clindamycin, More suitably, the formulations comprise about 0.050% to about 0.50% clindamycin, or about 0.080% to about 0.50% clindamycin, about 0.080% to about 0.40% clindamycin, about 0.080% to about 0.30% clindamycin, about 0.080% to about 0.20% clindamycin, about 0.08% to about 0.13% clindamycin, about 0.08% to about 0.12% clindamycin or about 0.080% clindamycin, about 0.085% clindamycin, about 0.090% clindamycin, about 0.095% clindamycin, about 0.096% clindtunycin, about 0.097% clindamycin, about 0.098% clindamycin, about 0.099% clindamycin, about 0.10% clindamycin, about 0.11% clindamycin, about 0.12% clindamycin, about 0.13% clindamycin, about 0.14% clindamycin, about 0.15% clindamycin, about 0.16% clindamycin, about 0.17% clindamycin, about 0.18% clindamycin, about 0.19% clindamycin, or about 0.20% clindamycin, including any value or range between these values.

In embodiments, suitably the topical formulations comprise about 0.010% to about 1.0% gentamicin, More suitably, the formulations comprise about 0.050% to about 0.50% gentamicin, or about 0.080% to about 0.50% gentarnicin, about 0.080% to about 0.40% gentarnicin, about 0.080% to about 0.30% gentamicin, about 0.080% to about 0.20% gentamicin, about 0.08% to about 0.13% gentamicin, about 0.08% to about 0.12% gentamicin or about 0.080% untamicin, about 0.085% gentamicin, about 0.090% gentamicin, about 0.095% gentamicin, about 0.096% gentamicin, about 0.097% gentamicin, about 0.098% gentarnicin, about 0.099% gentamicin, about 0.10% gentamicin, about 0.11% clindamycin, about 0.12% gentamicin, about 0.13% gentamicin, about 0.14% gentamicin, about 0.15% gentamicin, about 0.16% gentamicin, about 0.17% gentamicin, about 0.18% gentamicin, about 0.19% gentamicin, or about 0.20% gentamicin, including any value or range between these values.

In embodiments, suitably the topical formulations comprise about 1.0% to about 20.0% lawsonia inermis extract. More suitably, the formulations comprise about 5.0% to about 15.0% lawsonia inermis extract, or about 8.0% to about 15.0% lawsonia inermis extract, about 8.0% to about 14.0% lawsonia inermis extract, about 8.0% to about 13.0% lawsonia inermis extract, about 8.0% to about 13.0% lawsonia inermis extract, about 8.0% to about 12.0% lawsonia inerrnis extract, about 8.5% to about 11.5% lawsonia inermis extract or about 9.0% lawsonia inermis extract, about 9.1% lawsonia inermis extract, about 9.2% lawsonia inermis extract, about 9.3% lawsonia inermis extract, about 9.4% lawsonia inermis extract, about 9.5% lawsonia inermis extract, about 9.6% lawsonia inermis extract, about 9.7% lawsonia inermis extract, about 9.8% lawsonia inermis extract, about 9.9% lawsonia inermis extract, about 10.0% lawsonia inermis extract, about 10.1% lawsonia inermis extract, about 10.2% lawsonia inermis extract, about 10.3% lawsonia inermis extract, about 10.4% lawsonia inermis extract, about 10.5% lawsonia inermis extract, about 10.6% lawsonia inermis extract, about 10.7% lawsonia inermis extract, about 10.8% lawsonia inermis extract, about 10.9% lawsonia inermis extract, or about 11.0% lawsonia inermis extract, including any value or range between these values.

In embodiments, the formulations further comprise purified or de-ionized (DI) water making up the rest of the weight of the formulation.

Exemplary topical formulations include, but are not limited to, a cream, a lotion, a spray, a gel, a solution, a foam, an ointment and a mask, as well as other similar topical formulations known in the art.

Suitably the formulation is formulated as a spray, wherein the remainder of the formulation comprises water.

In further embodiments, the formulations can be formulated as a gel. In such embodiments, the formulations comprise carboxymethylcellulose sodium salt to form the gel. Other suitable excipients to form gels are well known in the art and can be readily used in the formulations described herein.

Suitably, the formulations exhibit a pH in the range of about 4.0 to about 7.0, more suitably about 4.5 to about 6.0, about 4.5 to about 5.5, or about 4.5, about 4.6, about 4.7, about 4.8, about 4.9, about 5.0, about 5.1, about 5.2, about 5.3, about 5.4 or about 5.5.

In embodiment, the formulations can comprise various non-active ingredients or excipients. Exemplary non-active ingredients or excipients are well known in the art. Such non-active ingredients include, but are not limited to, humectants, emollients, pH stabilizing agents, preservatives, chelating agents, gelling agents, thickening agents, emulsifiers, binders, buffers, carriers, anti-oxidants, etc.

Amounts of non-active agents, including those disclosed herein, are readily determinable by those of ordinary skill in the art. Generally, the amounts and ratios of the non-active agents are determined so as to provide the topical formulations with the desired consistency, stability, delivery characteristics and other properties of the formulation.

Exemplary non-active ingredients, include for example, dimethyl sulfoxide (DMSO), propylene glycol (PG), poly(ethylene glycol) 400 (400 molecular weight (MW)) (PEG 400), Transcutol, mineral oil, sterol alcohol, acetyl alcohol, Brij 21, Brij 721, emulsifying wax, monoglyceride (GMS), Isopropyl Myristate (IPM) Carbopol, Petrolatum,

The topical formulations can also comprise suitable preservatives, including for example, Methylparaben, Propylparaben and Benzyl Alcohol.

As described herein, suitably the formulations described throughout are useful in the treatment of wounds. The term “wound” is used throughout to mean a breach in the continuity of skin tissue which is caused by direct injury to the skin. Skin wounds are generally characterized by several classes: punctures, incisions, including those produced by a variety of surgical procedures (post-surgical wound), excisions, lacerations, abrasions, atrophic skin or necrotic wounds and bums, including large burn areas, and chronic wounds, such as for example, various ulcers such as decubitus ulcers (bed sores or other pressure ulcers), diabetic ulcers or venous stasis ulcers, etc.

In further embodiments, topical spray formulations for treatment of a wound are provided. Suitably, such formulations comprise about 0.0080% to about 0.0130% polymyxin B, about 0.080% to about 0.130% clindamycin, about 0.080% to about 0.130% gentamicin, about 8.0% to about 13.0% lawsortia inermis extract, and water making up the remainder of the formulation.

As described herein, the topical spray formulations suitably exhibit (have) a pH in the range of about 4.0 to about 7.0, more suitably about 4.5 to about 6.0, about 4.5 to about 5.5, or about 4.5, about 4.6, about 4.7, about 4.8, about 4.9, about 5.0, about 5.1, about 5.2, about 5.3, about 5.4 or about 5.5.

Also provided are topical gel formulations for treatment of a wound, Such formulations suitably comprise about 0.0080% to about 0.0130% polymyxin B, about 0.080% to about 0.130% clindamycin, about 0.080% to about 0.130% gentamicin, about 8.0% to about 13.0% lawsonia inermis extract and carboxymethylcellulose sodium salt (as well as water) to form the gel.

In embodiments, the topical formulations, including topical spray and gel formulations, suitably for treatment of a wound, comprise about 0.010% polymyxin B, about 0.10% clindamycin, about 0.10% gentamicin and about 10.0% lawsonia inermis extract (with the remainder being water).

The formulations described herein may also, in additional embodiments, comprise other active agents, including for example, additional antibiotics, various proteins, including growth factors, pain suppressants, anti-fungal agents, plant or tree extracts, etc.

In further embodiments, topical formulations are provided that consist of or consist essentially of the recited components at the recited amounts.

In formulations that consist essentially of the recited ingredients, such formulations specifically exclude other active agents, as the addition of such active agents would is considered a material alteration to such formulations and is thus excluded from such formulations that consist essentially of the recited active agents at the recited amounts.

Also provided are methods of treating a skin wound of a patient, including human and animal patients. Suitably, such methods comprise applying to the wound a topical formulation as described herein. For example, such methods comprise applying to the wound a formulation comprising about 0.0050% to about 0.050% polymyxin B, about 0.050% to about 0.50% clindamycin, about 0.050% to about 0.50% gentamicin, about 5.0% to about 15.0% lawsonia inermis extract and water.

In embodiments, the formulation is formulated as a spray and applied directly to the wound. That is, the spray formulation is sprayed directly onto the wound (as well as surrounding tissue).

In further embodiments, the formulation is formulated as a spray and is applied to a wound dressing. This wound dressing comprising the formulation is then applied to the wound.

As used herein, the term “wound dressing” is used to mean any suitable article for applying to and covering a wound, so as to facilitate healing. Exemplary wound dressings include for example, gauze, various fabrics, cloths, bandages, polymers, films, etc.

In further embodiments, as described herein, the formulation further comprises carboxymethylcellulose sodium salt and is formulated as a gel. Such gels can be directly applied to the wound or can be applied to a wound dressing that is then applied to the wound.

As described herein, suitably the topical formulations for use in the methods exhibit (have) a pH in the range of about 4.0 to about 7.0, more suitably about 4.5 to about 6.0, about 4.5 to about 5.5, or about 4.5, about 4.6, about 4.7, about 4.8, about 4.9, about 5.0, about 5.1, about 5.2, about 5.3, about 5.4 or about 5.5.

In further embodiments, the formulations for use in the methods described herein can consist of or consist essentially of the recited components.

Suitable dosing schemes tor applying the formulations described herein can be readily determined by those of ordinary skill in the art. In exemplary embodiments, the formulations are applied to the wound once a day, twice a day or three times a day, etc. The duration of the application is dependent on the condition of the wound, the severity of the wound, and the patient's response to the formulation, the patient's age, physical health, etc. Suitably the duration of application can be from about a few days, to several weeks, to months, if necessary.

As described herein, the methods are suitably used to treat patients exhibiting skin wounds that include, for example, punctures, incisions, including those produced by a variety of surgical procedures (post-surgical wound), excisions, lacerations, abrasions, atrophic skin or necrotic wounds and burns, including large burn areas, as well as chronic wounds, such as for example, various ulcers such as decubitus ulcers (bed sores or other pressure ulcers), diabetic ulcers or venous stasis ulcers, etc.

Also provided are methods of treating a skin wound comprising applying to the wound at least once a day a topical spray formulation as disclosed herein. Suitably, the formulation comprises about 0.0080% to about 0.0130% polymyxin B, about 0.080% to about 0.130% clindamycin, about 0.080% to about 0.130% gentamicin, about 8.0% to about 13.0% lawsonia inermis extract, and water.

In embodiments, the topical spray formulation is applied to a wound dressing, and then the dressing is applied to the wound. In other embodiments, the spray formulation can be applied directly to the wound,

As described herein, suitably the methods of treatment are used to treat chronic wounds, including for example, decubitus ulcers, diabetic ulcers or venous stasis ulcers. The methods can also be used to treat other wounds, including for example, post-surgical wounds.

The inventor has surprisingly found that application of the formulations described herein to various wounds dramatically shortens the healing time required. For example, wounds that typically take 3-4 months to heal are healed in a period of 4-6 weeks. In addition, chronic wounds that have been unhealed for periods of 1 year, 2 years and 20 years, have been healed in approximately 5 days, 10 days and 6 months, respectively, by application of the formulations described herein.

In addition, use of the formulations described herein in wound treatment generally does not produce necrotic tissue or require surgical debridement during the healing process.

These results demonstrate the unexpected and superior wound healing achieved by the methods and formulations described herein.

It will be readily apparent to one of ordinary skill in the relevant arts that other suitable modifications and adaptations to the methods and applications described herein can be made without departing from the scope of any of the embodiments. The following examples are included herewith for purposes of illustration only and are not intended to be limiting.

### EXAMPLES

### Example 1

### Treatment of Toe Amputation

A topical formulation comprising polymyxin B, clindamycin, gentamicin, lawsonia inermis extract and water, was applied to a wound dressing and then the dressing was applied to the amputated toe of a 68 year-old diabetic patient. The formulation comprised about 0.0080% to about 0.0130% polymyxin B, about 0.080% to about 0130% clindamycin, about 0.080% to about 0.130% gentamicin, about 8.0% to about 13.0% lawsonia inermis extract, and water, The formulation was applied to the wound once a day.

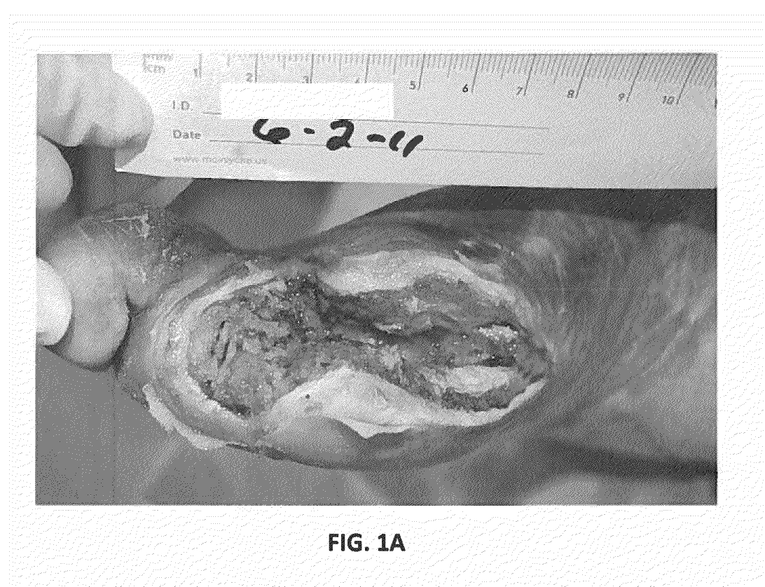

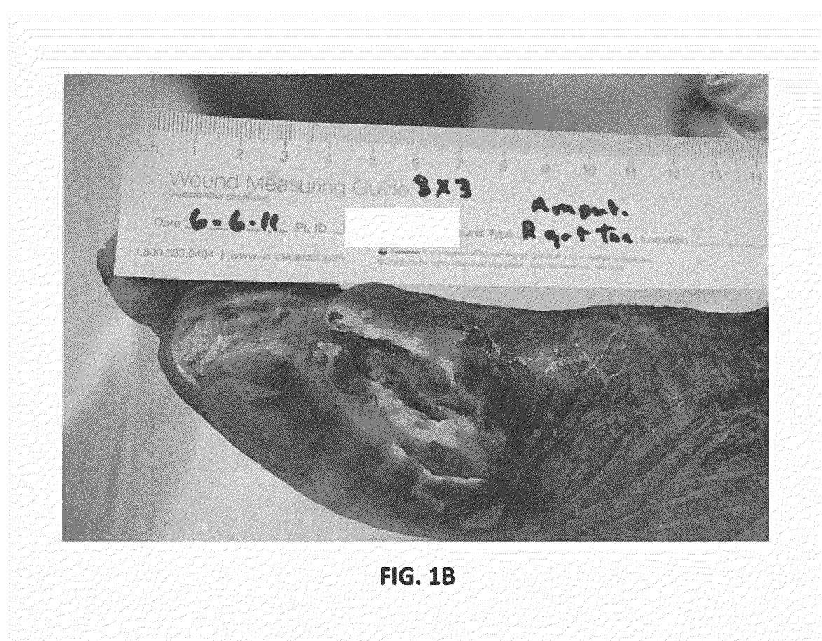

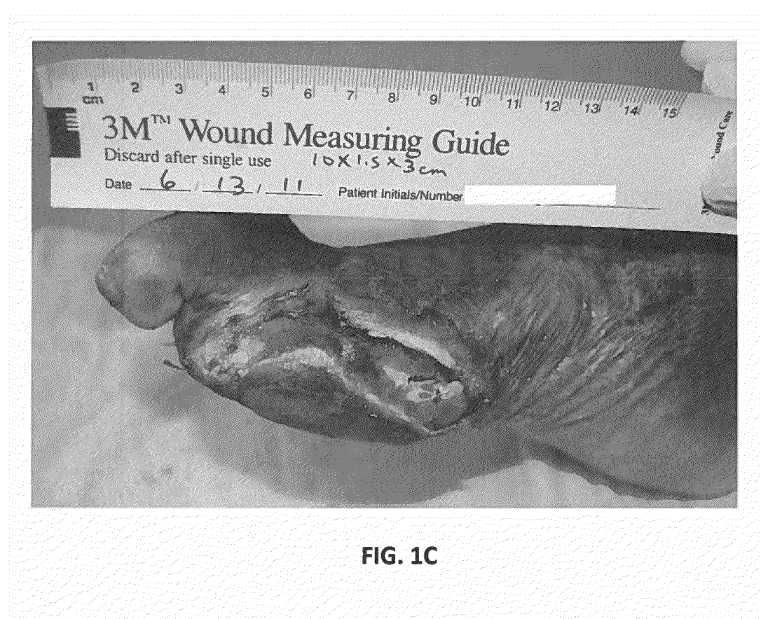

FIG. 1A shows the wound at the beginning of treatment.

FIG. 1B shows the wound at day 4 of treatment.

FIG. 1C shows the wound at day 11 of treatment,

The wound was fully healed by day 28 of treatment,

### Example 2

### Treatment of Hysterectomy Incision

A topical formulation comprising polymyxin B, clindamycin, gentamicin, lawsonia inermis extract and water, was applied to a wound dressing and then the dressing was applied to the abdomen of a female patient following hysterectomy. The formulation comprised about 0.0080% to about 0.0130% polymyxin B, about 0.080% to about 0.130% clindamycin, about 0.080% to about 0.130% gentamicin, about 8.0% to about 13.0% lawsonia inermis extract, and water. The formulation was applied to the wound once a day, so that that the entire surface of the wound was covered with a thin layer of the formulation.

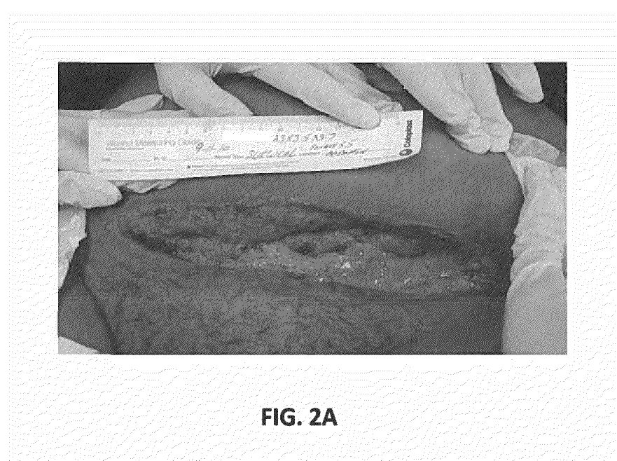

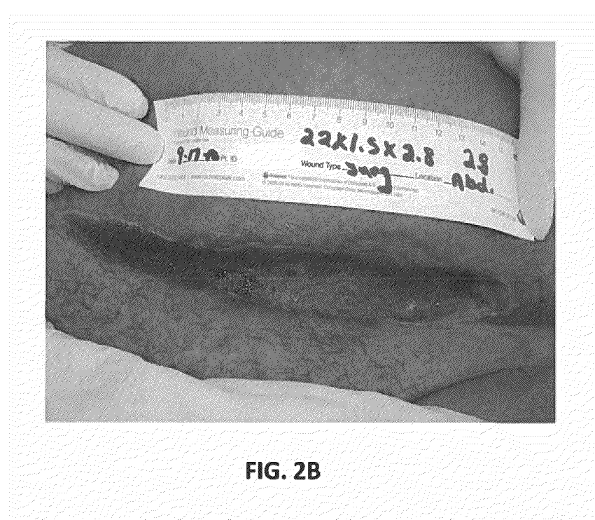

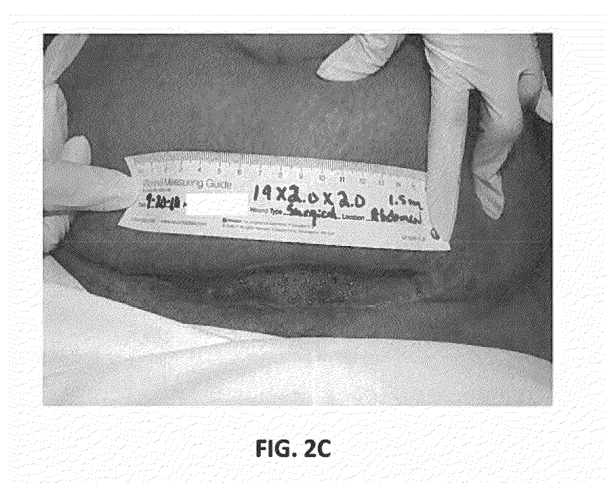

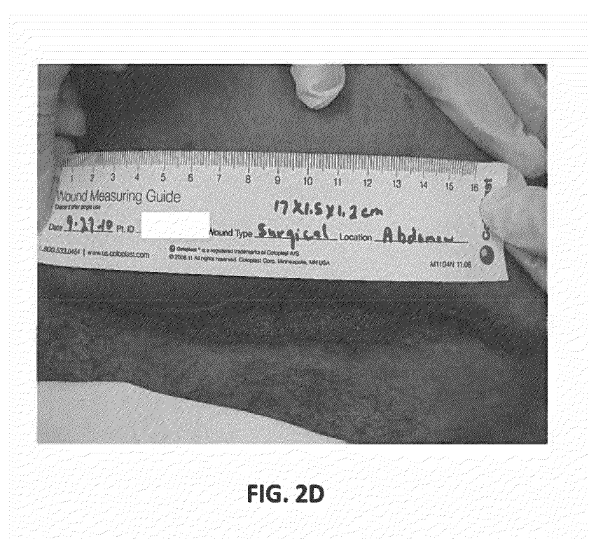

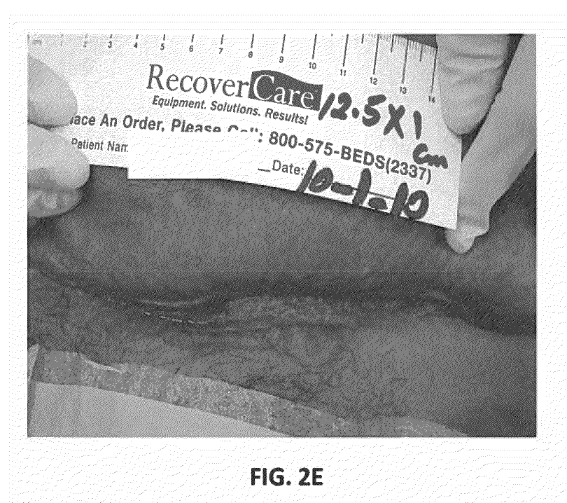

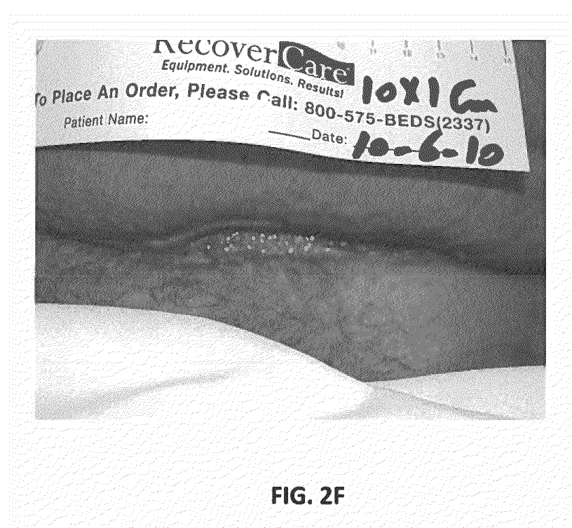

FIG. 2A shows the wound at the beginning of treatment.

FIG. 2B shows the wound at day 6 of treatment.

FIG. 2C shows the wound at day 9 of treatment.

FIG. 2D shows the wound at day 16 of treatment.

FIG. 2E shows the wound at day 20 of treatment.

FIG. 2F shows the wound at day 25 of treatment.

Table 1 below represents that approximate dimensions of the wound during the treatment cycle, demonstrating the rapid healing of the wound.

TABLE 1

| | Approximate Wound Dimensions |
|---|---|
| | (length × width × depth) in |
| Treatment Day | centimeters (cm) |
| 0 | 23 × 3.5 × 9.7 |
| 6 | 22 × 1.5 × 2.8 |
| 9 | 19 × 2.0 × 2.0 |
| 16 | 17 × 1.5 × 1.2 |
| 20 | 12.5 × 1 |
| 25 | 10 × 1 |

The wound healed in just over one month.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may he practiced otherwise than as specifically described.

What is claimed is:

1. A topical formulation comprising:
a) about 0.0050% to about 0.050% polymyth B;
b) about 0.050% to about 0.50% clindamycin;
c) about 0.050% to about 0.50% gentamicin;
d) about 5% to about 15% lawsonia inermis extract; and
e) water,

2. The topical formulation of claim 1, formulated as a spray.

3. The topical formulation of claim 1, further comprising carboxymethylcellulose sodium salt, formulated as a gel.

4. The topical formulation of claim 1, having a pH in the range of about pH 4.5 to about 5.5.

5. The topical formulation of claim 3, having a pH of about pH 5.0.

6. The topical formulation of claim 1, comprising:
a) about 0.0080% to about 0.0130% polymyxin B;
b) about 0.080% to about 0.130% clindamycin;
c) about 0.080% to about 0.130% gentamicin ;
d) about 8.0% to about 13.0% lawsonia inermis extract;
e) and water.

7. A topical spray formulation for treatment of a wound, comprising:
a) about 0.0080% to about 0.0130% polymyxin B;
b) about 0.080% to about 0.130% clindamycin;
c) about 0.080% to about 0.130% gentamicin ;
d) about 8.0% to about 13.0% lawsonia inermis extract; and
e) water.

8. The topical spray formulation of claim 7, having a pH of about 5.0.

9. A topical gel formulation for treatment of a wound, comprising:
a) about 0.0080% to about 0.0130% polymyxin B;
b) about 0.080% to about 0.130% clindamycin;
c) about 0.080% to about 0.130% gentamicin;
d) about 8,0% to about 13.0% lawsonia inermis extract;
3) carboxymethylcellulose sodium salt; and
f) water.

10. A method of treating a skin wound comprising applying to the wound a topical formulation comprising:
a) about 0.0050% to about 0.050% polymyxin B;
b) about 0.050% to about 0.50% clindamycin;
c) about 0.050% to about 030% gentamicin;
d) about 5.0% to about 15.0% lawsonia inermis extract; and
e) water.

11. The method of claim 10, wherein the formulation is formulated as a spray and applied directly to the wound.

12. The method of claim 10, wherein the formulation is formulated as a spray and applied to a wound dressing and then the dressing is applied to the wound.

13. The method of claim 10, wherein the formulation further comprises carboxymethylcellulose sodium salt and is formulated as a gel, and is applied directly to the wound.

14. The method of claim 10, wherein the formulation further comprises carboxymethylcellulose sodium salt and is formulated as a gel, and is applied to wound dressing and then the dressing is applied to the wound.

15. The method of claim 10, wherein the formulation has a pH in the range of about pH 4.5 to about 5.5.

16. The method of claim 15, wherein the formulation has a pH or about pH 5.0.

17. The method of claim 10, wherein the formulation comprises:
a) about 0.0080% to about 0.0130% polymyxin B;
b) about 0.080% to about 0.130% clindamycin;
c) about 0.080% to about 0.130% gentamicin;
d) about 8.0% is about 13.0% lawsonia inermis extract; and
e) water

18. The method of claim 10, wherein the formulation is applied to the wound at least once per day.

19. The method of claim 10, wherein the wound is a chronic wound.

20. The method of claim 19, wherein the wound is a decubitus ulcer, a diabetic ulcer or a venous stasis ulcer.

21. The method of claim 10, wherein the wound is a post-surgical wound.

22. A method of treating a skin wound comprising applying to the wound at least once a day a topical spray formulation comprising;
a) about 0.0080% to about 0.0130% polymyxin B;
b) about 0.080% to about 0.130% clindamycin;
c) about 0.080% to about 0.130% gentaimcin;
d) about 8.0% to about 13.0% lawsonia inennis extract; and
e) water,
wherein the applying comprises applying the spray tbratulation to a wound dressing and then applying the dressing to the wound.

23. The method of claim 22, wherein the formulation has a pH in the range of about pH 4.5 to about 5.5.

24. The method of claim 23, wherein the formulation has a pH or about pH 5.0.

25. The method of claim 21 wherein the wound is a chronic wound.

26. The method of claim 25, wherein the wound is a decubitus ulcer, a diabetic ulcer or a venous stasis ulcer.

27. The method of claim 22, wherein the wound is a post-surgical wound.
